(19) United States
(12) Patent Application Publication
(10) Pub. No.: US 2005/0009006 A1
(43) Pub. Date: Jan. 13, 2005

(54) High throughput monitoring chamber for testing drug effects on repolarization and conduction

(76) Inventors: Ofer Binah, Nofit (IL); Ira S. Cohen, Stony Brook, NY (US); Richard B. Robinson, Cresskill, NJ (US); Michael R. Rosen, New York, NY (US)

Correspondence Address: Cooper and Dunham LLP, 1185 Avenue of the Americas, New York, NY, 10036, US

(21) Appl. No.: 10/841,266

(22) Filed: May 7, 2004

Related U.S. Application Data
(60) Provisional application No. 60/469,492, filed on May 9, 2003.

(51) Int. Cl. C12Q 1/00; C12N 5/06

(52) U.S. Cl. 435/4; 435/353

(57) ABSTRACT

The invention provides a method for determining the effects of an agent on repolarization of cells in vitro, comprising stimulating the cells with an energy source and under conditions sufficient and for a time sufficient to depolarize the cells, measuring the QT interval of the electrical signals output by the cells in response to the stimulating step, contacting the cells with an agent, re-stimulating the cells with the same energy source and under the same conditions as the first stimulating step and for a time sufficient to depolarize the cells, measuring the QT interval of the electrical signals output by the cells in response to the second stimulating step, and comparing the results of the measuring taken after the first and second stimulating steps to determine whether the agent affects repolarization of cells. The present invention also provides a method for determining the effects of an agent on conduction of cells in vitro.

### CROSS-REFERENCE TO RELATED APPLICATIONS

This application claims priority of U.S. Provisional Application Ser. No. 60/469,492 filed on May 9, 2003, incorporated by reference herein.

### STATEMENT REGARDING SPONSORED RESEARCH OR DEVELOPMENT

The invention disclosed herein was made with Government support under NIH Grant Nos. HL-28958 and HL-53956 from the National Institutes of Health. Accordingly, the U.S. Government has certain rights in this invention.

### BACKGROUND OF THE INVENTION

Throughout this application, various publications are referenced to by numbers. Full citations may be found at the end of the specification immediately preceding the claims. The disclosures of these publications in their entireties are hereby incorporated by reference into this application in order to more fully describe the state of the art to those skilled therein as of the date of the invention described and claimed herein.

The present invention relates to a high throughput monitoring chamber for testing drug effects on repolarization and conduction.

Some pharmacological compounds can negatively affect cardiac repolarization or conduction, thereby triggering cardiac dysrhythmias in individuals. Early detection and screening of these potentially harmful compounds will advance drug discovery and development. Currently, only low-throughput screens, utilizing isolated tissue, intact animal, or cell-culture systems, are available. These existing screens are relatively expensive and can only generate 10's of data points a day. The present invention is directed towards providing a reliable, high-throughput, cell based assay capable of screening thousands of compounds a month in order to evaluate their effects on cardiac repolarization and conduction such that potential proarrhythmic and therapeutic effects can be identified.

### SUMMARY OF THE INVENTION

The present invention provides a method for determining the effects of an agent on repolarization of cells in vitro. The steps of the method comprise stimulating the cells with an energy source and under conditions sufficient and for a time sufficient to depolarize the cells, measuring the QT interval of the electrical signals output by the cells in response to the stimulating step, contacting the cells with an agent, re-stimulating the cells with the same energy source and under the same conditions as the first stimulating step and for a time sufficient to depolarize the cells, measuring the QT interval of the electrical signals output by the cells in response to the second stimulating step, and comparing the results of the measuring taken after the first and second stimulating steps to determine whether the agent affects repolarization of cells.

The present invention also provides a method for determining the effects of an agent on conduction of cells in vitro, comprising stimulating the cells with an energy source and under conditions sufficient and for a time sufficient to depolarize the cells, measuring the spike duration of the electrical signals output by the cells in response to the stimulating step, contacting the cells with an agent, re-stimulating the cells with the same energy source and under the same conditions as the first stimulating step and for a time sufficient to depolarize the cells, measuring the spike duration of the electrical signals output by the cells in response to the stimulating step, and comparing the results of the measuring taken after the first and second stimulating steps to determine whether the agent affects conduction of the cells.

### BRIEF DESCRIPTION OF THE FIGURES

FIG. 1A-F show a data acquisition system and methods for construction of activation maps;

FIG. 1A shows the recording electrodes layout on which neonatal rat ventricular myocytes (NRVM) were plated;

FIG. 1B shows NRVM plated around 4 recording electrodes;

FIG. 1C shows the inter-spike interval of a 7-day old culture;

FIG. 1D shows a fast sweep-speed trace of a unipolar electrogram recorded from one electrode site;

FIG. 1E shows the calculation of the local activation time (LAT) from the electrogram recorded at one electrode site;

FIG. 1F shows an activation map constructed from the LAT at each electrode, the scale of the map being between 0 and 35 ms; and

FIG. 2 shows two graphs illustrating the effects of an I_{Kr}-blocking drug, E4031, on repolarization relative to a control, and showing in particular the prolonged repolarization occurring in the presence of the drug.

### DETAILED DESCRIPTION OF THE INVENTION

The present invention provides a method for determining the effects of an agent on repolarization of cells in vitro. The steps of the method comprise stimulating the cells with an energy source and under conditions sufficient and for a time sufficient to depolarize the cells, measuring the QT interval of the electrical signals output by the cells in response to the stimulating step, contacting the cells with an agent, re-stimulating the cells with the same energy source and under the same conditions as the first stimulating step and for a time sufficient to depolarize the cells, measuring the QT interval of the electrical signals output by the cells in response to the second stimulating step, and comparing the results of the measuring taken after the first and second stimulating steps to determine whether the agent affects repolarization of cells.

The cells of the above-described method may be cardiac myocytes disaggregated from a species having an I_{Kr} current, such as neonatal rat cardiac myocytes.

Alternatively, the cells may be transfected with a gene, such as the HERG gene, or the cells may be stem cells transfected with a gene, such as the HERG gene.

The agent of the above-described method may be a drug.

The step of stimulating of the above-described method may comprise stimulating the cells with an energy source and under conditions sufficient and for a time sufficient to depolarize the cells in a testing well.

The testing well of the above-described method may have an inner diameter of 3 mm by 3 mm with one 150×30 μm stimulating electrode and one 30 μm diameter electrode placed at opposite ends of the well. The electrodes may have titanium-nitrite gold contacts and may be insulated with silicone nitride.

The present invention also provides a method for determining the effects of an agent on conduction of cells in vitro, comprising stimulating the cells with an energy source and under conditions sufficient and for a time sufficient to depolarize the cells, measuring the spike duration of the electrical signals output by the cells in response to the stimulating step, contacting the cells with an agent, re-stimulating the cells with the same energy source and under the same conditions as the first stimulating step and for a time sufficient to depolarize the cells, measuring the spike duration of the electrical signals output by the cells in response to the stimulating step, and comparing the results of the measuring taken after the first and second stimulating steps to determine whether the agent affects conduction of the cells.

The cells of the above-described method may be cardiac myocytes disaggregated from a species having an I_{Kr} current, such as neonatal rat cardiac myocytes.

Alternatively, the cells may be transfected with a gene, such as the HERG gene, or the cells may be stem cells transfected with a gene, such as the HERG gene.

The agent of the above-described method may be a drug.

The step of stimulating of the above-described method may comprise stimulating the cells with an energy source and under conditions sufficient and for a time sufficient to depolarize the cells in a testing well.

The testing well of the above-described method may have an inner diameter of 3 mm by 3 mm with one 150×30 μm stimulating electrode and one 30 μm diameter electrode placed at opposite ends of the well. The electrodes may have titanium-nitrite gold contacts and may be insulated with silicone nitride.

As used herein, the term “repolarization” means the process whereby a membrane, cell, or fibre, after depolarization, is polarized again, with positive charges on the outer and negative charges on the inner surface.

As used herein, the term “depolarize” means to reduce to an unpolarized condition.

As used herein, the term “QT interval” means the time from electrocardiogram Q wave to the end of the T wave corresponding to electrical systole.

As used herein, the term “HERG gene” means the human ether-a-go-go related gene which generates the I_{Kr} current that is recorded from isolated cardiac myocytes.

As used herein, the term “I_{Kr} current” means the delayed rectifier potassium current.

As used herein, the term “cardiac myocytes” means myocytes derived from muscle or conductive tissue of a heart, either isolated or in culture, and capable of initiating a current.

Methods explaining the above detailed description are set forth below.

The present invention provides a high throughput monitoring chamber for testing drug effects on repolarization and conduction.

Preparation of Cultured Myocytes

Extracellular matrix collagen type I from calfskin (Sigma C-8919) was diluted 1:10 in 0.1M acetic acid and 0.5 ml of the solution was applied to the MEA for 3-4 hours at room temperature. Prior to myocyte plating, the MEA was rinsed with PBS.

Cultures of neonatal rat ventricular myocytes (NRVM) were prepared as previously described (9), with some modifications. Ventricles from 1-2 day old Sprague-Dawley rats were dissociated enzymatically at room temperature using the protease RDB (Cat # 300-0, IIBR, Ness-Ziona, Israel). The enzyme was diluted 1:100 in phosphate buffered saline (PBS) containing glucose (1 mg/ml) and antibiotics (100 U/ml penicillin, 100 mg/ml streptomycin). The myocytes were then collected by 10 minute centrifugation (1600 rpm) at the end of a 10 minute cycle of digestion. The tissue fragments were dispersed after eight to ten cycles. Each cell pellet was then rinsed with Ham's F10 (Cat # 01-090-1A, Biological Industries, Beit-Haemek, Israel) and resuspended in fresh Ham's F10. The pooled cells were filtered through a stainless steel grid, centrifuged, and resuspended in growth medium (Ham's F10 supplement with 5% fetal calf serum, 5% horse serum, 100 U/ml penicillin, 100 mg/ml streptomycin, 1 mM CaCl₂ (up to a total concentration of 1.3 mM), and 50 mg/100 ml bromodeoxyuridine (BrdU) (Sigma, B-5002)). The myocytes were then placed in a monitoring chamber onto the bottom of a testing well at a density of 2-3×10⁶ myocytes/ml. The chamber contains between 24 to 96 such wells. The cultures were maintained in a humidified incubator with an atmosphere of 5% CO₂ and 95% air at 37° C.

Data Acquisition System, Culture Stimulation, and Electrical Activity Recording

The monitoring chamber is a PC-based data acquisition system (Multi Channel Systems, Reutlingen, Germany), consisting of multi-electrode arrays (MEAs), pre- and filter-amplifiers, a data acquisition board and software. The MEA consists of a 50×50 mm glass substrate, in the center of which is embedded a 1.4×1.44 m matrix of 60 titanium-nitride, gold contact, 30 μm diameter electrodes insulated with silicone nitride, with an interelectrode distance of 200 μm respectively (note that there are no electrodes at the corner of the matrix). Cultures are stimulated using one of the four pairs of stimulating electrodes (250 μm×50 μm) located 2 mm from each of the four external rows of recording electrodes. Data is recorded at 10 kHz with 12-bit precision. To permit data recording, the MEA is removed from the incubator, constantly perfused with fresh culture medium, and saturated with a gas mixture consisting of 5% CO₂ and 95% air at 37° C.

The MEA mapping system used is compatible with the paradigm that the spacing between recording electrodes must be larger than the electrodes themselves (13). Eason and Malkin used a finite element model with modified Fitzhugh-Nagumo kinetics, in which the electrodes were represented as isopotential surfaces of varying width and spacing ratios (center-to-center spacing divided by the electrode diameter, spacing ratio—SR) (14), and simulated the ability of a single electrode to detect the conduction velocity (among other parameters) due to a passing wavefront. The propagation velocity for the reference stimulation (in the absence of electrodes) was 37 cm/sec (a value compatible with the conduction velocities measured), and the detected propagation velocities for all electrode sizes (10-100 μm) were within 10% of the reference velocity for all SR's>1.0. Thus, for SR of 6.7 (200 μm/30 μm) of the electrode matrix, the conduction velocities are reproducibly measured.

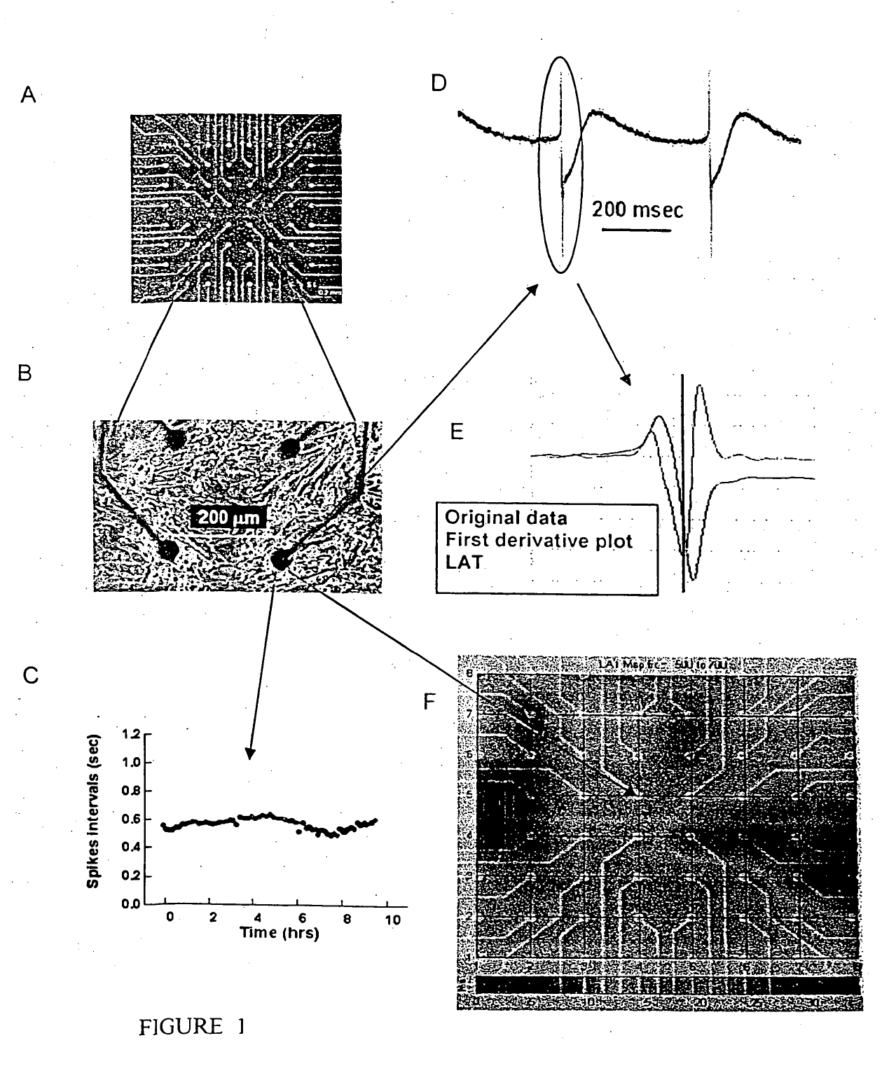

FIGS. 1A-F show the data acquisition system and methods for the construction of activation maps. The Multi-Electrode Array (MEA) system was utilized to record electrical activity from neonatal rat ventricular (NRVM) cultures. FIG. 1A shows the recording electrodes layout on which NRVM myocytes were plated with electrode diameter of 30 μm diameter and inter-electrode distance of 200 μm. FIG. 1B is a photograph depicting NRVM plated around 4 recording electrodes. FIG. 1C is an inter-spike interval of a spontaneously firing 7-day old culture. The recording was performed for 10 hours under regular culture conditions, while the spontaneous activity was relatively stable. FIG. 1D shows a fast sweep-speed trace of a unipolar electrogram recorded from one electrode site. FIG. 1E is the calculation of the local activation time (LAT) from the electrogram recorded at one electrode site. The blue trace is the electrogram, the red trace is the electrogram first derivative and the green vertical line is the minima of the differentiated signal, denoting LAT. FIG. 1F is an activation map constructed from the LAT at each electrode. The isochronal map was constructed using linear interpolation between the electrodes, calculated by means of the MATLAB software. The recordings electrode matrix is superimposed on the colored map. The scale of the map is between 0 and 35 ms.

Data were filtered using a bidirectional Butterworth fourth-order low-pass digital filer to obtain zero phase distortion with a cutoff frequency of 2 kHz. The filtered signal was differentiated digitally to determine the local activation time (LAT) at each electrode. The calculation also provided the maximal voltage change of the QRS (dV/dtmax). The color-coded activation maps were constructed by interpolating the LAT values for the sites between the electrodes and extrapolating the LAT values for the four corners of the MEA matrix. Activation maps were plotted using the Matlab standard two-dimensional plotting function (pcolor) (Matlab 5.3; Mathworks Inc.)

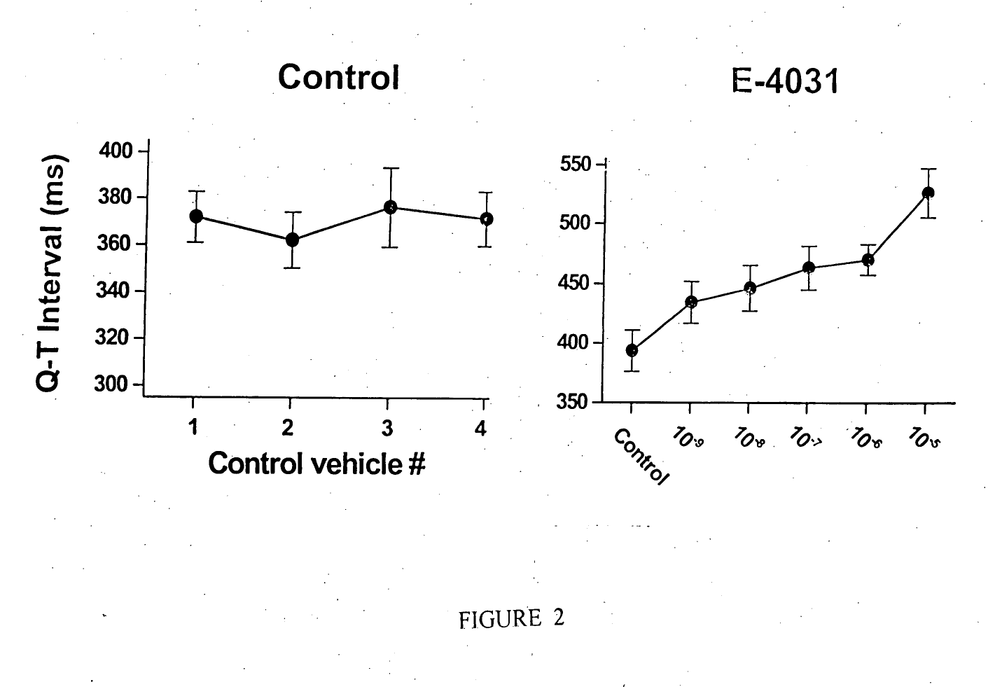

Alternatively, cell lines or stem cells transfected with the HERG and other genes may be used instead of the cardiac myocytes. When drugs are to be assayed, the chamber is moved to a standard microscope for stimulation and recording of electrical signals. The electrogram spike and QT interval are recorded. Drug is then added to each well in graded amounts and the effects of the drug on spike duration, reflecting conduction, and QT interval, reflecting repolarization, are recorded by the aforementioned PC-based data acquisition system (FIG. 2). The standard method of use would include a control and three concentrations of the drug, but the system can support an array of possibilities with more controls and more drug concentrations.

Although a preferred embodiment of the invention is described, the invention is not so limited, as variations and modifications will occur to those skilled in the art.

The scope of the invention is determined by way of the appended claims.

References

1. Fast V G. and Kleber A G.: Microscopic conduction in cultured strands of neonatal rat heart cells measure with voltage-sensitive dye. Circ. Res. Vol. 73, 1993, pages 914-925.
2. Thomas S P, et al.: Synthetic strands of neonatal mouse cardiac myocytes: Structural and electrophysiological properties. Circ. Res. Vol. 87, 2000, pages 467-473.
3. Fast V G, et al.: Anistropic activation spread in heart cell monolayers assessed by high-resolution optical mapping. Role of tissue discontinuities. Circ. Res. Vol. 79, 1996, pages 115-127.
4. Kimura H, et al.: Reversible inhibition of gap junctional intercellular communication, synchronous contraction, and synchronism of intracellular Ca²⁺ fluctuation in cultured neonatal rat cardiac myocytes by heptanol. Exp. Cell Res. 1995, pages 348-356.
5. Beardslee M A, et al.: Rapid turnover of connexins 43 in the adult rat heart. Circ. Res. Vol. 83, 1998, pages 629-635.
6. Wang T L, et al.: Regulation of connexins 43 gene expression by cyclical mechanical stretch in neonatal rat cardiomyocytes. Biochem. Biophys. Res. Commun. Vol. 267, 2000, pages 551-557.
7. Zhuang J, et al.: Pulsatile stretch remodels cell-to-cell communication in cultured myocytes. Circ. Res. Vol. 87, 2000, pages 316-322.
8. Oyamada M, et al.: The expression, phosphorylation, and localization of connexins 43 and gap-junction intercellular communication during the establishment of a synchronized contraction of cultured neonatal rat cardiac myocytes. Exp. Cell Res. Vol. 212, 1994, pages 351-358.
9. Rubin Y, et al.: The effect of furosemide on calcium ion concentration in myocardial cells. Cell Calcium Vol. 18, 1995, pages 135-139.
10. Cain M E, et al.: Signal-averaged electrocardiography. J. Am. Coll. Cardiol. Vol. 27, 1996, pages 238-249.
11. Spach M S and Dolber P C: Relating extracellular potentials and their derivatives to anistropic propagation at a microscopic level in human cardiac muscle. Circ. Res. Vol. 58, 1986, pages 356-371.
12. Bayly P V, et al.: Estimation of conduction velocity vector fields from epicardial mapping data. IEEE Trans Biomed. Eng. Vol. 45, 1988, pages 563-571.
13. Witkowski F X, et al.: In vivo estimation of cardiac transmembrane current. Circ. Res. Vol. 72, 1993, pages 424-439.
14. Eason J C and Malkin R A: A simulation study evaluating the performance of high-density electrode arrays on myocardial tissue. IEEE Trans Biomed. Eng. Vol. 47, 2000, pages 893-901.
15. Spach M S, et al.: Electrophysiological effects of remodeling cardiac gap junctions and cell size. Experimental and model studies of normal cardiac growth. Circ. Res. Vol. 86, 2000, pages 302-311.
16. Saffitz J E, et al.: Connexin expression and turnover. Circ. Res. Vol. 86, 2000, pages 723-728.
17. Zuppinger C, et al.: Dynamics of early contact formation in cultured adult rat cardiomyocytes studied by N-cadherin fused to green fluorescent protein. J. Mol. Cell Cardiol. Vol. 32, 2000, pages 539-555.
18. Rook M B, et al.: Gap junction formation and functional interaction between neonatal rat cardiomyocytes in culture. A correlative physiological and ultrastructural study. J. Membr. Biol. Vol. 118, 1990, pages 179-192.
19. Litchenberg W H, et al.: The rate and anisotropy of impulse propagation in the postnatal terminal crest are correlated with remodeling of Cx43 gap junction pattern. Cardiovasc. Res. Vol. 45, 2000, pages 379-387.
20. Netzer R et al.: Screening lead compounds for QT interval prolongation. Drug Discovery Today Vol. 6, No. 2, 2001, pages 78-84.
21. Meiry, G. et al.: Evolution of Action Potential Propagation Repolarization in Cultured Neonatal Rat Ventricular Myocytes. Journal of Cardiovascular Electrophysiology Vol. 12, No. 11, 2001, pages 1269-1277.

1. A method for determining the effects of an agent on repolarization of cells in vitro, comprising:
(a) stimulating the cells with an energy source and under conditions sufficient and for a time sufficient to depolarize the cells;
(b) measuring the QT interval of the electrical signals output by the cells in response to the stimulating step (a);
(c) contacting the cells with an agent;
(d) re-stimulating the cells with the same energy source and under the same conditions as step (a) and for a time sufficient to depolarize the cells;
(e) measuring the QT interval of the electrical signals output by the cells in response to the stimulating step (d); and
(f) comparing the results of the measuring in steps (b) and (e) to determine whether the agent affects repolarization of the cells.

2. The method of claim 1, wherein the cells are cardiac myocytes disaggregated from a species having an I_{Kr} current.

3. The method of claim 2, wherein the cardiac myocytes are neonatal rat cardiac myocytes.

4. The method of claim 1, wherein the cells are transfected with a gene.

5. The method of claim 4, wherein the gene is the HERG gene.

6. The method of claim 1 wherein the cells are stem cells.

7. The method of claim 6, wherein the stem cells are transfected with a gene.

8. The method of claim 7, wherein the gene is the HERG gene.

9. The method of claim 1, wherein the agent is a drug.

10. The method of claim 1, wherein the step of stimulating comprises stimulating the cells with an energy source and under conditions sufficient and for a time sufficient to depolarize the cells in a testing well.

11. The method of claim 10, wherein the testing well has an inner diameter of 3 mm by 3 mm.

12. The method of claim 11, wherein the testing well comprises two electrodes placed at opposite ends of the well.

13. The method of claim 12, wherein the electrodes comprise one 150×30 μM stimulating electrode and one 30 μm diameter electrode.

14. The method of claim 13, wherein the electrodes have titanium-nitrite gold contacts and are insulated with silicone nitride.

15. A method for determining the effects of an agent on conduction of cells in vitro, comprising:
(a) stimulating the cells with an energy source and under conditions sufficient and for a time sufficient to depolarize the cells;
(b) measuring the spike duration of the electrical signals output by the cells in response to the stimulating step (a);
(c) contacting the cells with an agent;
(d) re-stimulating the cells with the same energy source and under the same conditions as step (a) and for a time sufficient to depolarize the cells;
(e) measuring the spike duration of the electrical signals output by the cells in response to the stimulating step (d); and
(f) comparing the results of the measuring in steps (b) and (e) to determine whether the agent affects conduction of the cells.

16. The method of claim 15, wherein the cells are cardiac myocytes disaggregated from a species having an I_{Kr} current.

17. The method of claim 16, wherein the cardiac myocytes are neonatal rat cardiac myocytes.

18. The method of claim 15, wherein the cells are transfected with a gene.

19. The method of claim 18, wherein the gene is the HERG gene.

20. The method of claim 15, wherein the cells are stem cells.

21. The method of claim 20, wherein the stem cells are transfected with a gene.

22. The method of claim 21, wherein the gene is the HERG gene.

23. The method of claim 15, wherein the agent is a drug.

24. The method of claim 15, wherein the step of stimulating comprises stimulating the cells with an energy source and under conditions sufficient and for a time sufficient to depolarize the cells in a testing well.

25. The method of claim 24, wherein the testing well has an inner diameter of 3 mm by 3 mm.

26. The method of claim 25, wherein the testing well comprises two electrodes placed at opposite ends of the well.

27. The method of claim 26, wherein the electrodes comprise one 150×30 μM stimulating electrode and one 30 μm diameter electrode.

28. The method of claim 27, wherein the electrodes have titanium-nitrite gold contacts and are insulated with silicone nitride.
